(19) United States
(12) Patent Application Publication
(10) Pub. No.: US 2016/0030169 A1
(43) Pub. Date: Feb. 4, 2016

(54) DUAL FRAME STENT AND VALVE DEVICES AND IMPLANTATION

(71) Applicant: AORTIC INNOVATIONS, LLC, Boca Raton, FL (US)

(72) Inventor: Ali Shahriari, Boca Raton, FL (US)

(21) Appl. No.: 14/774,718

(22) PCT Filed: Mar. 7, 2014
(86) PCT No.: PCT/US2014/022144

§ 371 Date: Sep. 11, 2015

Related U.S. Application Data
(60) Provisional application No. 61/780,242, filed on Mar. 13, 2013.
(60) Provisional application No. 61/817,118, filed on Apr. 29, 2013.

(51) Int. Cl. A61F 2/24; A61F 2/90; A61F 2/844

(52) U.S. Cl. CPC A61F 2/2418; A61F 2/2409; A61F 2/844; A61F 2/90; A61F 2220/0016; A61F 2250/001; A61F 2250/0069

(57) ABSTRACT

A device for placement in a biological tissue fluid conduit includes an outer frame, multiple stabilizer arms, an inner frame, and a valve. The outer frame circumferentially surrounds a longitudinal axis and defines an interior passageway. The outer frame has an interior side facing the longitudinal axis and passageway, and an exterior side opposite the interior side. The stabilizer arms extend outward from the outer frame. The inner frame is positioned in the passageway and circumferentially surrounds the longitudinal axis with at least a longitudinal portion of the inner frame circumferentially surrounded by the outer frame. The inner frame has an exterior side facing the interior side of the outer frame. The valve is housed by the inner frame and is configured to restrict fluid flow through the passageway in at least one of two longitudinal directions.

### CROSS-REFERENCE TO RELATED APPLICATION

This application is a 371 National Phase application of PCT application no. PCT/US14/22144, titled “Dual Frame Stent and Valve Devices and Implantation,” filed on Mar. 7, 2014, and claims the benefit of priority of U.S. provisional patent application No. 61/780,242, titled “Transcatheral Mitral Valve and Delivery System,” filed on Mar. 13, 2013, and the benefit of priority of U.S. provisional patent application No. 61/817,118, titled “Device for Transcatheter Mitral Valve Repair and Replacement,” filed on Apr. 29, 2013, both of which are incorporated herein in entirety by these references.

### TECHNICAL FIELD

The present disclosure relates to a device that may be used as a transcathetar mitral valve replacement (TCMVR), including repair of mitral regurgitation (MR), mitral stenosis (MS), and also for the treatment of tricuspid valve disorders.

### BACKGROUND

Surgical mitral valve repair/replacement (sMVR) is a proven and lasting therapy for patients with symptomatic mitral regurgitation (MR) and mitral stenosis (MS). Although highly successful with acceptable risk in younger patients with degenerative valvular conditions, sMVR is a risky procedure with high morbidity and mortality in higher risk patients. Alternative transcatheter approaches such as mitral clip, transcatheter annuloplasty through the coronary sinus, neochordal attachment, and a few designs of transcatheter mitral valve replacements (TCMVR) have been attempted but with variable and inconsistent rates of success.

### SUMMARY

This Summary is provided to introduce in a simplified form concepts that are further described in the following detailed descriptions. This Summary is not intended to identify key features or essential features of the claimed subject matter, nor is it to be construed as limiting the scope of the claimed subject matter.

In at least one embodiment, a device for placement in a biological tissue fluid conduit includes an outer frame, multiple stabilizer arms, an inner frame, and a valve. The outer frame circumferentially surrounds a longitudinal axis and defines an interior passageway. The outer frame has an interior side facing the longitudinal axis and passageway, and an exterior side opposite the interior side. The stabilizer arms extend outward from the outer frame. The inner frame is positioned in the passageway and circumferentially surrounds the longitudinal axis with at least a longitudinal portion of the inner frame circumferentially surrounded by the outer frame. The inner frame has an exterior side facing the interior side of the outer frame. The valve is housed by the inner frame and is configured to restrict fluid flow through the passageway in at least one of two longitudinal directions.

In at least one example, the inner frame has an unexpanded state, in which a gap is defined between the exterior side of the inner frame and the interior side of the outer frame, and an expanded state, in which the gap is reduced or increased.

In at least one example, the inner frame has an unexpanded state, in which a gap is defined between the exterior side of the inner frame and the interior side of the outer frame, and an expanded state, in which the exterior side of the inner frame engages the interior side of the outer frame. In at least one example, the inner frame includes a stent that is expandable from the unexpanded state to the expanded state. In at least one example, the inner frame and outer frame are self-expanding. In at least one example, at least one of the inner frame and outer frame is balloon expandable.

In at least one example, the outer frame includes: a tubular body section surrounding the longitudinal axis and having a longitudinal first end extending in a longitudinal first direction and a longitudinal second end extending in a longitudinal second direction opposite the first direction; and a flange having a longitudinal first end joined to the second end of the body section and a longitudinal second end extending radially away from the longitudinal axis and widening in the second direction.

In at least one example, the outer frame includes a tubular body section surrounding the longitudinal axis and having a longitudinal first end extending in a longitudinal first direction and a longitudinal second end extending in a longitudinal second direction opposite the first direction. Hooks may extend from the first end of the body section at least radially away from the longitudinal axis. The hooks may extend radially away from the longitudinal axis and in the second direction.

In at least one example, each stabilizer arm includes a spring urged away from the longitudinal axis. In at least one example, each stabilizer arm includes a base secured to the outer frame and a beam extending from the base and urged away from the longitudinal axis. At least one beam may be configured as a cantilever spring urged away from the longitudinal axis.

Each stabilizer arm may include barbs. In at least one example, each stabilizer arm includes a base secured to the outer frame, a tip opposite the base, and a beam from which barbs extend, the beam extending from the base to the tip. The barbs of at least one stabilizer arm are directed at least partially toward the tip thereof

In at least one example, each stabilizer arm includes an S-shaped portion.

In at least one embodiment, a device for placement in a biological tissue fluid conduit includes: an outer frame circumferentially surrounding a longitudinal axis and defining an interior passageway, the outer frame having an interior side facing the longitudinal axis and passageway, and an exterior side opposite the interior side; and multiple stabilizer arms extending outward from the outer frame.

In at least one example, the device further includes: an inner frame dimensioned to be positioned at least partially in the passageway circumferentially surrounding the longitudinal axis with at least a longitudinal portion of the inner frame circumferentially surrounded by the outer frame, the inner frame having an exterior side for facing the interior side of the outer frame; and a valve housed by the inner frame and configured to restrict fluid flow through the passageway in at least one of two longitudinal directions. The valve and its frame may be positioned in a supra-annular location.

In at least one example, the device further includes: an expandable stent for positioning at least partially in the passageway circumferentially surrounding the longitudinal axis, the stent having an unexpanded state in which the stent is movable within the outer frame, and an unexpanded state in which an exterior side of the stent engages the interior side of the outer frame. In at least one example, the stent and outer frame are each self-expanding. In at least one example, the stent is balloon expandable and the outer frame is self-expanding.

In at least one example, the outer frame includes: a tubular body section surrounding the longitudinal axis and having a longitudinal first end extending in a longitudinal first direction and a longitudinal second end extending in a longitudinal second direction opposite the first direction; and a flange having a longitudinal first end joined to the second end of the body section and a longitudinal second end extending radially away from the longitudinal axis and widening in the second direction. In at least one example, each stabilizer arm comprises an S-shaped portion.

### BRIEF DESCRIPTION OF THE DRAWINGS

The previous summary and the following detailed descriptions are to be read in view of the drawings, which illustrate particular exemplary embodiments and features as briefly described below. The summary and detailed descriptions, however, are not limited to only those embodiments and features explicitly illustrated.

FIG. 1 is a perspective view of a dual-frame device for valve replacement or repair according to at least one embodiment.

FIG. 2 is an exploded view of the device of FIG. 1.

FIG. 3 is an elevation view of the outer component of the device of FIG. 1.

FIG. 4 is a plan view of the outer component of FIG. 3.

FIG. 5 is an elevation view of the inner frame and valve component of the device of FIG. 1.

FIG. 6 is an elevation view of the device of FIG. 1.

FIG. 7 is an plan view of the device of FIG. 1.

FIG. 8 is a partial cutaway view of a heart with the device of FIG. 1 implanted therein.

FIG. 9 is an elevation view of another embodiment of an outer component.

FIG. 10 is a plan view of the outer component of FIG. 9.

FIG. 11 is an elevation view of another embodiment of a device for valve replacement or repair.

### DETAILED DESCRIPTIONS

While the concepts of the present disclosure are susceptible to various modifications and alternative forms, specific exemplary embodiments thereof have been illustrated by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

Terms representing anatomical references, such as anterior, posterior, medial, lateral, superior, inferior, caudal, cranial, etcetera, may be used throughout the specification in reference to the implants and surgical instruments described herein as well as in reference to the patient's natural anatomy. Such terms have well-understood meanings in both the study of anatomy. Use of such anatomical reference terms in the written description and claims is intended to be consistent with their well-understood meanings unless noted otherwise. For example, the term “cranial” refers to the direction that is generally toward the head of the patient, and the term “caudal” refers to the direction that is generally toward the feet of the patient.

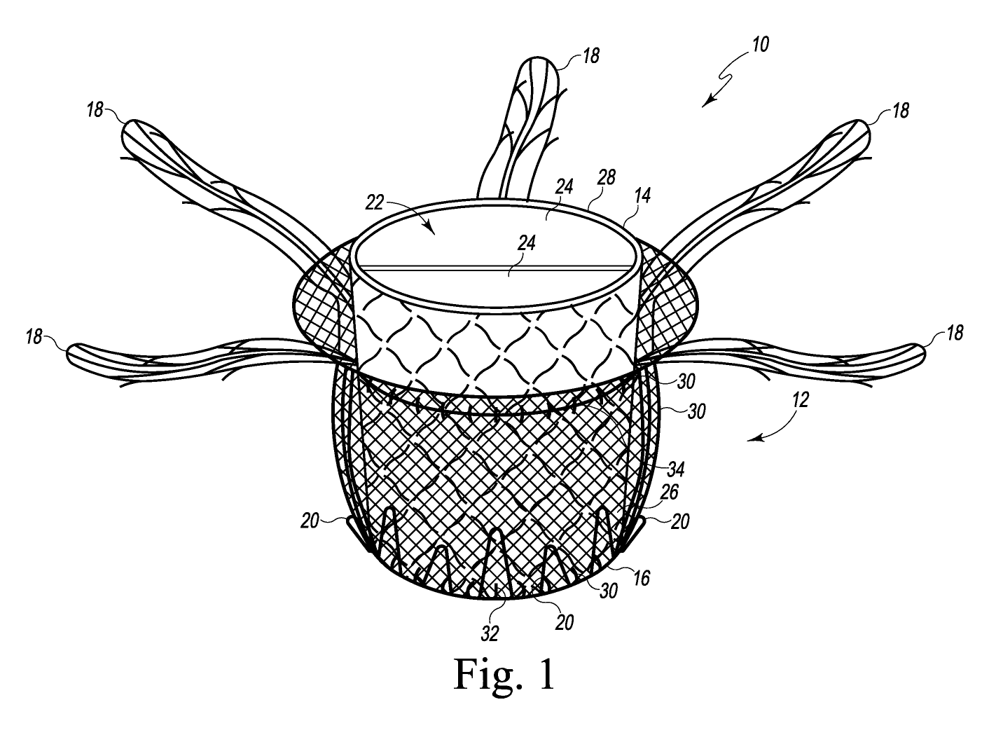

These descriptions begin with an overview of a device 10 overall and its use before further describing various particular components in detail. Referring to FIGS. 1-8, the device 10 for transcatheter mitral valve repair and replacement is shown. The device 10 is intended for the treatment, repair, and/or replacement of a damaged mitral valve as often occurs during mitral regurgitation (MR) and/or mitral stenosis (MS). The device may also be used to treat tricuspid valve disorders. The device 10 may be deployed and implanted through the apex of the heart or transvenously via the femoral or jugular veins. Further, the device 10 offers effective sealing to prevent paravalvular leaks from the device 10 and also prevents obstruction of the blood outflow tract in the left ventricle.

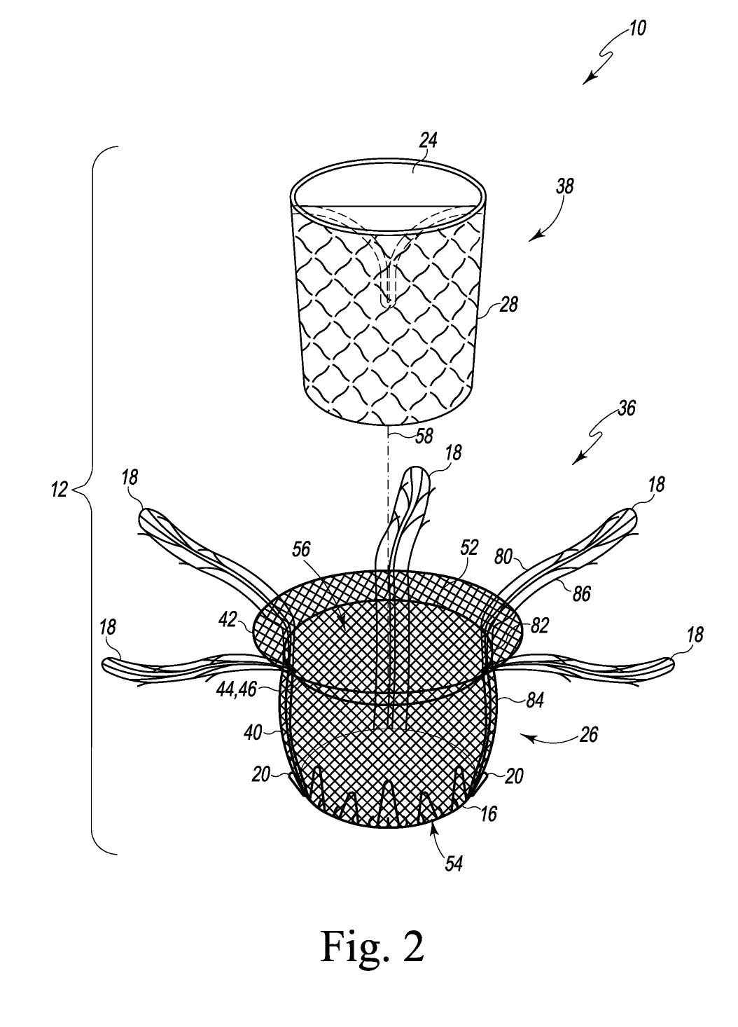

As shown in FIG. 1, the device 10 includes a dual-frame 12 that extends from a cranial end 14 to a caudal end 16. When the device 10 is implanted into a patient's heart, blood flow is regulated through a valve 22 carried by the dual frame 12. In FIG. 2, the dual-frame character of the device 10 is illustrated by separation of an inner frame 28 from an outer frame 26. In FIG. 1, the inner frame 28 is shown in its operable position within the outer frame 26, and when assembled as such the inner frame 28 and outer frame 26 constitute the dual-frame 12.

In these descriptions, embodiments of a dual frame are described as placement structures for valve replacement or repair of the mitral valve in a human heart. However, the outer frame 26 more generally may be described as a stabilizing outer frame for placement of the inner frame 28, which may be generally described as an expandable stent, with or without the valve 22. Furthermore, the device 10, with or without the valve 22, may be generally described as a device for placement in a biological tissue fluid conduit.

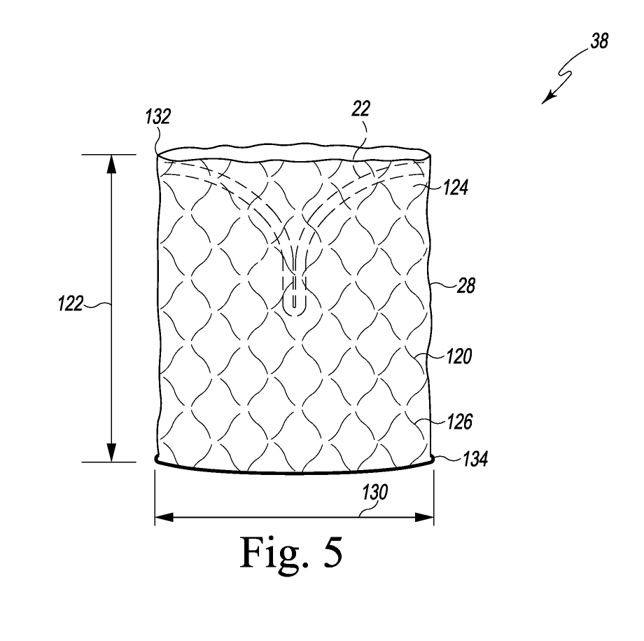

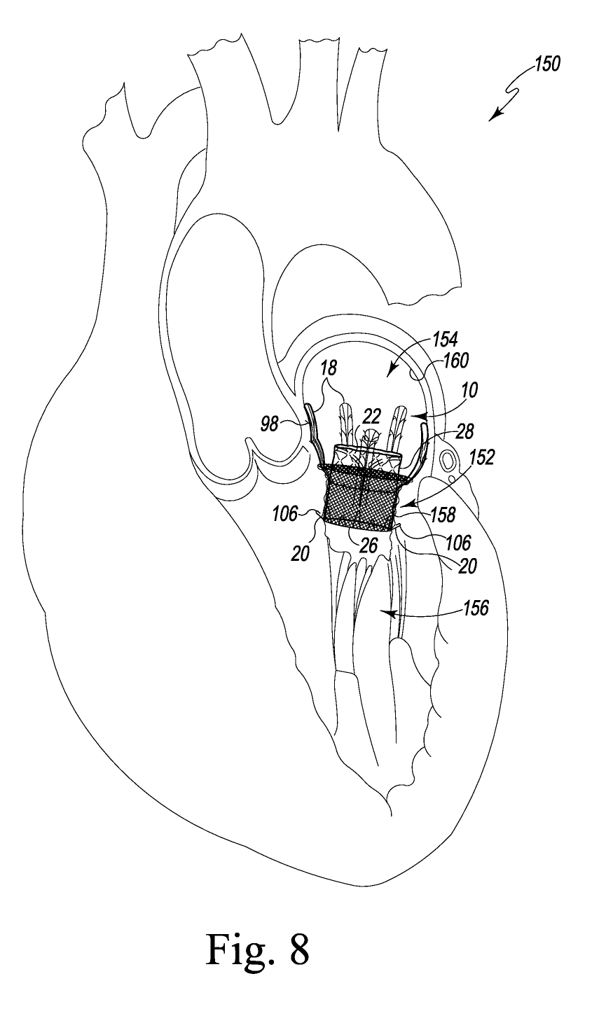

In the illustrated embodiment, however, the inner frame 28 carries and houses the valve 22, which is positioned generally at the cranial end 14 of the dual frame 12 in the drawings embodiment. In other embodiments, the valve 22 may be positioned at the caudal end 16 of the dual frame 12 or somewhere between the cranial and caudal ends. The valve 22 may be constructed from treated bovine pericardium or other suitable proven biological or synthetic material. The valve 22 may be configured, for example, as bicuspid, tricuspid or quadracuspid. In the illustrated embodiment, the valve 22 is configured as a bicuspid valve including a pair of leaflets 24. The leaflets 24, as shown in FIGS. 2 and 5, cusp toward the caudal direction with respect to a longitudinal axis 58 (FIG. 2) of the device, such that the leaflets 24 are generally more permissive of blood flow in the caudal direction. In the installation scenario of FIG. 8, in which the device 10 is implanted into a patient's heart, the valve 22 replaces the patient's natural mitral valve and permits fluid (i.e., blood) to selectively pass from the left atrium to the left ventricle. As such, forces experienced by the device 10 due to blood pressure are expected to be greater toward the cranial direction than the caudal direction, for example at least due to the arrangement of the leaflets 24 which restrict flow in the cranial direction.

Toward the cranial end of the device 10, a number of stabilizing arms 18 are secured to the dual-frame 12 and extend outwardly therefrom. The stabilizing arms 18 engage arterial and heart tissue at the cranial end of the device, stabilizing the device 10 as the heart beats. At the caudal end of the device 10, a number of barbs or hooks 20 extend outward to engage arterial and heart tissue, further stabilizing the device at the caudal end.

With particular additional regard to the dual frame 12, in the illustrated embodiment, the dual-frame 12 of the device 10 includes the outer frame 26 and the inner frame 28 that is secured to the outer frame 26. The outer frame 26 and inner frame 28 are each self-expanding in at least one embodiment. In at least one other embodiment, one or more of the outer frame and inner frame is balloon expandable. In the illustrative embodiment, the inner frame 28 is secured to the outer frame 26 via a plurality of connectors such as stitches 30 or other mechanical connectors or adhesive. As shown in FIG. 1, the stitches 30 include a circumferential row 32 of stitches 30 at the caudal end 16 of the dual frame 12 and another circumferential row of stitches 30 positioned between the cranial end 14 and the caudal end 16. It should be appreciated that in other embodiments soldering, welding or other fasteners may be used to secure the inner frame 28 to the outer frame 26. In other embodiments, the caudal portion of the inner frame 28 may be free floating at the caudal end 16 of the device 10.

As shown in FIGS. 1-2, the arms 18 and the barbs 20 are secured to the outer frame 26 to form an outer component 36 of the device 10. The inner frame 28 and the valve 22 cooperate to form an inner component 38 of the device 10. As shown in FIG. 2, the outer frame 26 includes an elongated tubular body section 40 circumferentially surrounding the longitudinal axis 58, and a cone-shaped annular flange section 42, which has a caudal end 46 joined to the cranial end 44 of the body section 40. The flange section 42 extends radially outwardly and longitudinally in the cranial direction from the body section 40 such that the flange section 42 widens in the cranial direction. In the illustrative embodiment, the tubular body 40 and cone-shaped annular flange 42 are formed as a single monolithic component. It should be appreciated that in other embodiments the body 40 and flange 42 may be formed separately and later joined to assemble the outer frame 26 by welding, stitching, mechanical fasteners, or other techniques.

In at least one embodiment, the outer frame 26 is formed from mesh wire. The mesh wire may be formed from a metallic material, such as, nitinol, stainless steel, or other implant grade metallic material. It should be appreciated that in other embodiments the outer frame 26 may be formed from a polymeric material.

In the illustrated embodiment of FIGS. 2-4, the outer frame 26 is uncovered. In other embodiments, the outer frame 26 may be covered. Such covering may coat each wire of the primary mesh structure, and/or may sheath the other frame overall among the wires of the mesh. The outer frame 26 may be covered with low profile polyester, polytetrafluoroethylene (PTFE), expanded PTFE (ePTFE), polyethylene glycol (PEG) polymer, Dyneema, polyethylene terephthalate (PET) and/or other nonporous covering materials and biologically inert or acceptable materials such that fluid is prevented from contacting the primary mesh structure and/or passing through the frame 26. The outer frame frame 26 may be also covered with low-profile Dacron or other synthetic material. It should also be appreciated that all or part of the frame 26 may be covered with microfibers, hydrogel or other sealing material to aid in the sealing function of the device 10. The above-mentioned coatings and coverings may be mono-layered or multi-layered laminates upon the primary mesh structure.

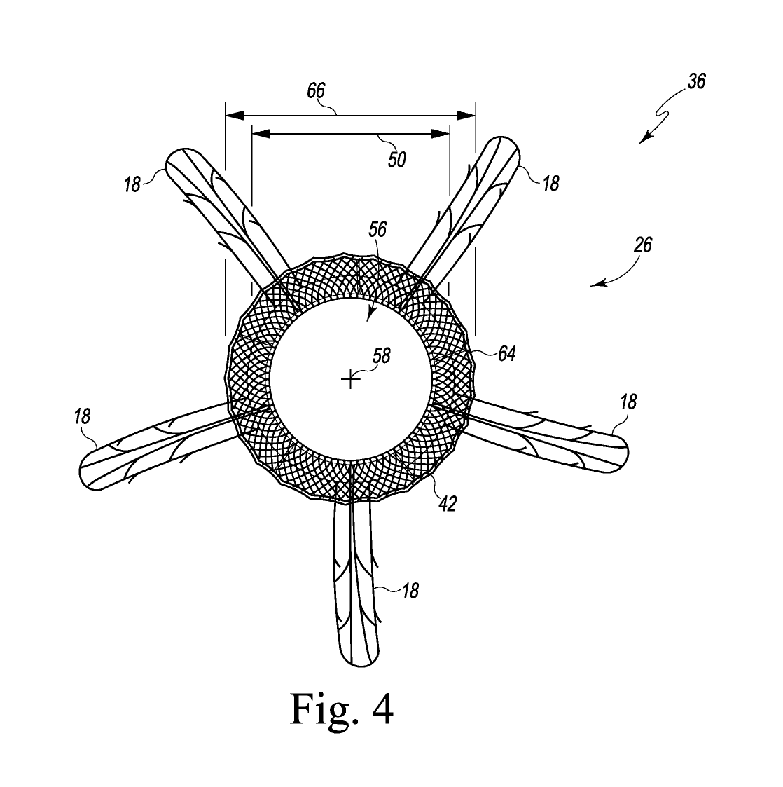

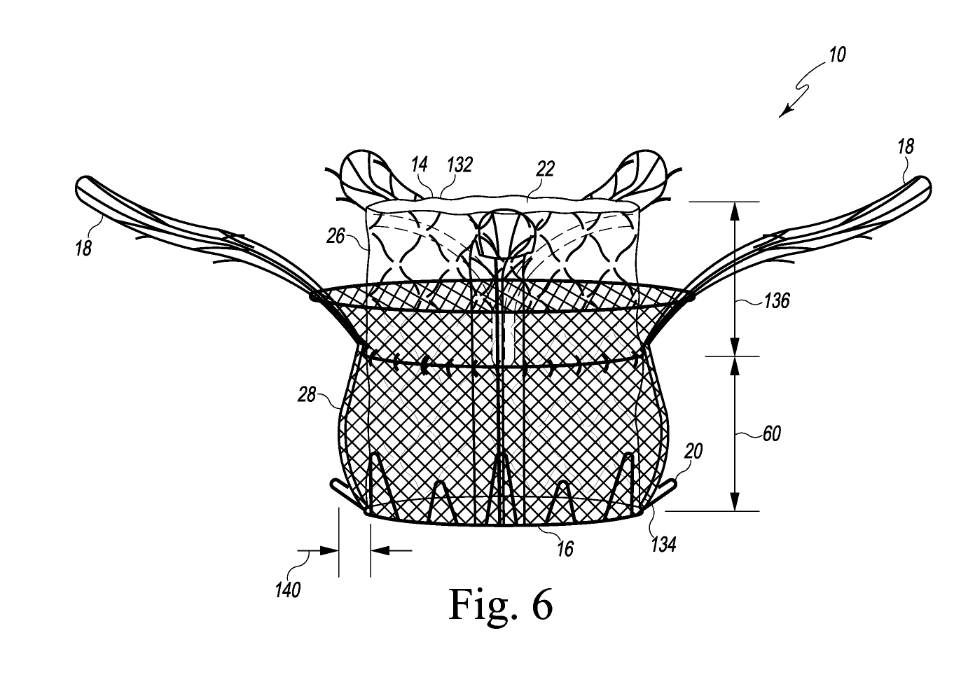

In the illustrative embodiment, the body section 40 of the outer frame 26 has a circular cross-section when viewed longitudinally (FIG. 4). The caudal end 16 of the device 10 is defined by the caudal end of the tubular body section 40 of the outer frame 26. At least a caudal end longitudinal portion of the inner frame 28 is positioned within and circumferentially surrounded by the outer frame 26 in FIG. 1. Due to the cranial direction extension of the inner frame 26 relative to the outer frame 12, the cranial end of the inner frame 26 defines the cranial end 14 of the dual frame as shown in FIG. 6.

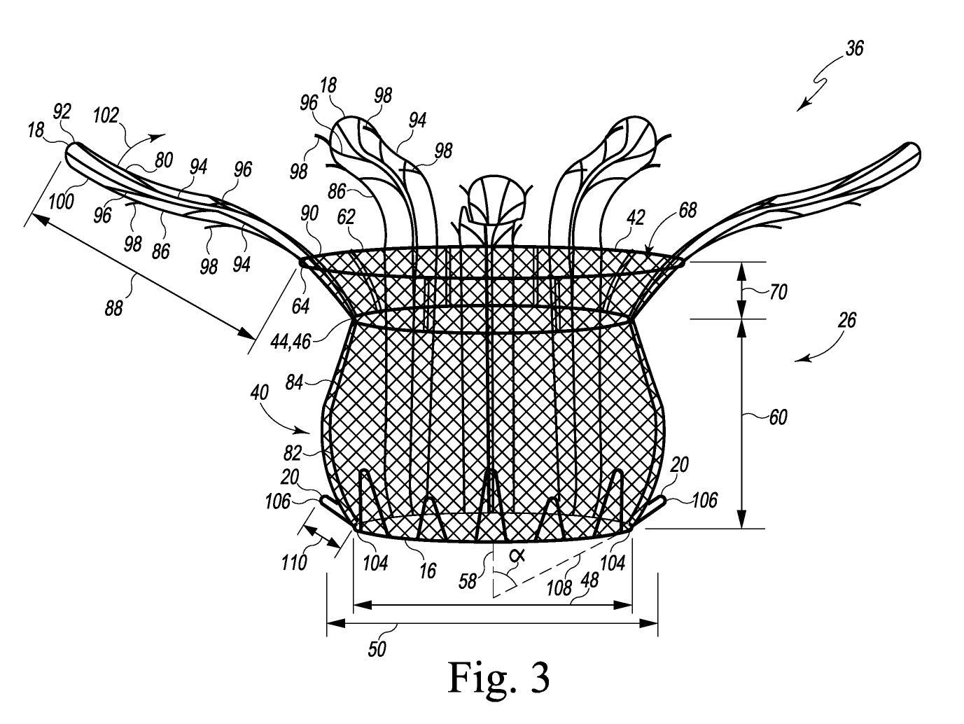

As shown in FIGS. 2-3, the tubular body section 40 bows outward between its cranial end 44 and caudal end. Thus, the body section 40 has a lesser outer diameter 48 at the caudal end 16 and the cranial end 44 and a greater outer diameter 50 at the bulging waist of the body section 40 between the ends 16 and 44, the greater diameter 50 being greater than the lesser diameter 48. In at least one embodiment, the lesser diameter 48 is equal to 27 millimeters and the greater diameter 50 is equal to 32 millimeters. In other embodiments, the outer diameter 48 may be equal to 26 millimeters. Additionally, it should be appreciated that in other embodiments the diameter at the caudal end 16 of the body section 40 and dual frame may be greater than or smaller than the diameter at the cranial end 44 of the body section 40. In other embodiments, the diameters 48, 50 may be equal such that the tubular body section 40 is essentially cylindrical.

As shown in FIG. 2, the body section 40 of the outer frame 26 has a cranial opening 52 defined at the cranial end 44 and a caudal opening 54 defined at the caudal end 16. The diameters of the openings 52, 54 correspond to the diameters 48 of the ends 16, 44. As such, in the illustrative embodiment the openings 52, 54 are equal in diameter. In other embodiments, diameter of the opening 52 may be less than the diameter of the opening 54. A passageway 56 extends between the openings 52, 54. As described in greater detail below, the inner frame 28 extends longitudinally outwardly from the passageway 56 in the cranial direction when assembled with the outer frame 26.

The passageway 56 of the body section 40 extends along the longitudinal axis 58 (FIG. 2). As shown in FIG. 3, the body section 40 has a height 60 defined along the axis 58 between the ends 16 and 44. In the illustrative embodiment, the height 60 is equal to 20 millimeters. In other embodiments, the height 60 may be in the range of 15 to 25 millimeters. It should be appreciated that in other embodiments the body section 40 may be longer or shorter depending on the size and configuration of the patient's heart.

As described above, the outer frame 26 also includes an annular flange 42 that extends outwardly from the body section 40 and circumferentially surrounds the longitudinal axis 58. With further regard to the flange 42, and as shown in FIG. 3, the annular flange 42 has a caudal end 46 connected to the cranial end 44 of the body section 40 to an opposite end 62. In the illustrative embodiment, the end 62 of the annular flange 42 defines a rim 64 having a diameter 66 (see FIG. 4) greater than the diameters 48, 50 of the body section 40. The diameter 66 is illustratively 37 mm but may be greater than or less than 37 mm depending on the patient's anatomy. As shown in FIG. 3, the annular flange 42 defines an annular chamber 68 that is connected to the passageway 56 of the body section 40. As shown in FIG. 3, the annular chamber 68 has an opening defined by the rim 64 that is greater than the openings 52, 54 of the body section 40.

The annular flange 42 has a height 70 defined along the longitudinal axis 58 between the ends 46 and 62. In the illustrative embodiment, the height 70 is equal to 5 millimeters. It should be appreciated that in other embodiments the annular flange 42 may be longer or shorter depending on the size and configuration of the patient's heart.

As described above, the outer component 36 of the device 10 also includes a number of hooks 20 secured at the caudal end 16. The barbs 20 are configured to stabilize the device 10 in the native annulus defined between the atrium and the ventricle. In the illustrative embodiment, each barb 20 is formed from a metallic material, such as, nitinol, stainless steel, or other implant grade metallic material. It should be appreciated that in other embodiments the barbs 20 may be formed from a polymeric material. Each barb 20 has a base 104 (FIG. 3) that is secured to the outer frame 26 via welding, but it should be appreciated that in other embodiments the barbs 20 may be secured to the outer frame 26 by a suture technique or other fastening means.

Each barb 20 of the device 10 extends in a partially cranial direction from its base 104 to a tip 106 spaced apart from the frame 26. As shown in FIGS. 2-4, each barb 20 is triangular in shape. A longitudinal axis 108 extends through the tip 106 and the base 104 of each barb 20. As shown in FIG. 3, an angle a is defined between the longitudinal axis 108 of each barb 20 and the longitudinal axis 58 of the outer component 36. In the illustrative embodiment, the magnitude of the angle a is 45 degrees. In other embodiments, the magnitude of the angle a may be in the range of 30 to 75 degrees.

Each barb 20 extends a length 110 between its base 104 and tip 106. In the illustrative embodiment, the length 110 is equal to 5 mm. In other embodiments, the length 110 may be in the range of 5 to 15 mm. Additionally, in other embodiments, the barbs 20 may be formed from cranially directed, oblique, blunt- tipped triangles or rods with alternating height and angles against the longitudinal axis 58 of the outer component 36. In one such embodiment, the rods may have a height of 5 and 10 mm in an alternating fashion. The rods may also have alternating angles formed with the longitudinal axis 58, preferentially between 30 and 45 degrees. In other embodiments, the barbs may be secured to an inner aspect of the inner frame 28.

As described above, the self-expanding outer frame 26 cooperates with a number of stabilizing arms 18 and a number of hooks 20 to form an outer component 36 of the device 10. As shown in FIG. 2, the device 10 includes five stabilizing arms 18, and each arm 18 is configured to engage the atrial wall of the heart. It should be appreciated that in other embodiments the device 10 may include less than or more than five stabilizing arms 18. For example, in some embodiments, the device 10 may include four arms 18; in other embodiments, the device 10 may include six arms 18 to stabilize the device 10 in the atrium. Due to the flexibility of the stabilizing arms 18, which follow the contours of host tissue upon placement, for example as shown in FIG. 8, the tips of the arms 18 may or may not extend further in the cranial direction than the cranial end 14 (FIG. 6) of the dual frame.

The stabilizing arms 18 bear mutual resemblance in the drawings such that any particular arm 18 may be described. The arm 18 is formed by a wire frame 80 that includes a number of longitudinal wires 94 and a number of traverse wires 96 that strengthen each beam 86. The wire frame 80 is open-cell and uncovered in the illustrated embodiment, thereby avoiding potential obstruction of a patient's pulmonary veins. In the illustrative embodiment, the wire frame 80 of each arm 18 is formed from a metallic material, such as, nitinol, stainless steel, or other implant grade metallic material. It should be appreciated that in other embodiments the wire frame 80 may be formed from a polymeric material.

The wire frame 80 includes a base 82 secured to the outer frame 26 and a beam 86 extending outwardly from the base 82. Each base may be secured to the outer surface 84 of the outer frame 26, but other arrangements are within the scope of the drawings and these descriptions. The base 82 of each arm 18 may secured to the outer frame 26 via welding and/or stitches 30, but it should be appreciated that in other embodiments the bases 82 may be secured to the outer frame 26 by a suture technique or other fastening means. Whatever the attachment, the base 82 has an inward facing side facing the longitudinal axis and outward facing side opposite the inward facing side. Following the curvature of the arm 18, the beam 86 can be described as having an inward side continued from the inward facing of the base 82 without ambiguity even as the beam may be curved away from the longitudinal axis near the caudal end 14 of the device 10. In keeping with that convention, the inward side of the beam 86 in FIGS. 2-3 generally faces a cranial direction in the drawings, while the opposing outward side 100 (FIG. 3) of the beam 86 generally faces the caudal direction.

As shown in FIGS. 2-3, the beam 86 of each stabilizing arm 18 extends from an end 90 positioned adjacent to the annular flange 42 to a tip 92. A length 88 is defined between the end 90 and the tip 92. As shown on the left in FIG. 3, the length 88 of each beam 86 is equal to approximately 25 mm. In the illustrative embodiment, the tip 92 of each beam 86 is relatively blunt to lessen the risk of injury to the atrial wall.

In the illustrated embodiment, the beam 86 is formed or configured to act as a cantilever spring. In FIGS. 1-4, each arm 18 is positioned in an unflexed or unloaded position. It should be understood the arms are flexible and, upon implantation into a host biological structure, may follow and engage host tissue contours as shown in FIG. 8. When a beam 86 is moved or flexed toward the longitudinal axis 58, as indicated by arrow 102 in FIG. 3, the beam 86 is configured to resist the movement and exert a force in the direction opposite the arrow 102. In that way, when the device 10 is unsheathed in a patient's heart, the beams 86 are urged away from longitudinal axis 58 until the beams 86 and the barbs 98 engage biological tissue such as an atrial wall (FIG. 8).

While the arms 18 are flexible and may follow and engage host tissue contours as shown in FIG. 8, each beam 86 can nonetheless be described as S-shaped (FIGS. 2-3) in its unloaded configuration without external forces or structures flexing the arm 18 or beam 86 from the unloaded configuration. In particular, in following the curvature of the beam 86 from the end 90 or base 82 (FIG. 3) to the tip 92, the beam 86 first curves outward toward the outward side 100, then inward in the direction 102 away from the outward side 100 such that the tip 92 is curved inward relative to an adjacent portion of the beam 86.

The arm 18 of the device 10 also includes a plurality of hooks or barbs 98 for engaging heart or arterial tissue. As shown in FIGS. 2-4, the barbs 98 extend outwardly from the outward surface 100 of each beam 86 along the length 88. The barbs 98 upon any particular beam 86 are directed at least partially toward the tip 92 and away from the base 82. In the illustrated embodiment, the barbs 98 extend away from the outer frame 26 to maximize each arms engagement with tissue, particularly to resist movement of the device 10 in the cranial direction. In other embodiments, barbs 98 and extend in alternating directions toward and away from the outer frame 26 and in the cranial and caudal directions. Each barb 98 is configured to engage the wall of the atrium of the patient's heart when the device 10 is implanted.

Under the expectation that forces experienced by the device 10 due to blood pressure may be greater toward the cranial direction than the caudal direction, both the stabilizing arms 18 and hooks 20 advantageously extend at least somewhat in the cranial direction with respect to the longitudinal axis 58 of the device 10, particularly when the beams 86 of the arms are flexed in the direction 102 (FIG. 3) from their unloaded configurations to engage arterial and heart tissue as shown in FIG. 8. Thus, the expected greater forces toward the cranial direction are countered by the engagement of the stabilizing arms 18 and hooks 20 with arterial and heart tissue. Both the stabilizing arms 18 and hooks 20 extend radially outward and longitudinally toward the cranial direction to facilitate maximum engagement with arterial and heart tissue by hooking action. As the heart beats, the leaflets 24 of the valve 22 (FIGS. 1 and 5) cyclically open under blood pressure in the caudal direction, permitting blood flow along the longitudinal axis 58 in the caudal direction. Subsequently, under blood pressure in the cranial direction, the leaflets 24 close to restrict blood flow in the cranial direction thus imparting cranially directed longitudinal force upon the device 10 which is countered by the stabilizing arms 18 and hooks 20.

Advantageously, barbs 98 extend outwardly from the outward surface 100 of each beam 86 along the length 88 thereof to engage tissue. The barbs 98 upon any particular beam 86 are advantageously directed at least partially toward the tip 92 and away from the base 82 to maximize tissue engagement particularly with regard to resisting movement of the device 10 in the cranial direction (upward in FIG. 8).

As shown in FIG. 5, the inner frame 28 houses the valve 22. The inner frame 28 in at least one embodiment is configured as a balloon-expandable tubular stent 120 that has a length 122 of approximately 25 mm defined between its cranial end 132 and caudal end 134. In other embodiments, the stent 120 may be longer or shorter depending on, for example, the patient's anatomy. In the illustrated embodiment of FIG. 1, the inner frame 28 is stitched to the outer frame, fixing their relative positions. In other embodiments, the inner frame is movable within the outer frame in an unexpanded configuration of the inner frame. In such embodiments, upon expansion of the inner frame to an expanded configuration, the inner frame engages the outer frame, fixing their relative positions. In at least one example, upon expansion of the inner frame to an expanded configuration, the inner frame bears outward force upon the outer frame causing further expansion of the outer frame and increased engagement with a surrounding host tissue structure.

The stent 120 is tubular and is constructed of a metallic material, such as, nitinol, stainless steel, or other implant grade metallic material, in an open-cell configuration. It should be appreciated that in other embodiments the stent 120 may be formed from a polymeric material and may be formed in, for example, a Z-stent configuration. As shown in FIG. 5, the stent 120 has a cylindrical outer surface 124, but in other embodiments the stent 120 may have an oval cross-section. In the illustrative embodiment, the outer surface 124 of the stent 120 is covered with low-profile polyester, PTFE, ePTFE, PET or other nonporous biologically inert covering material 126 that prevents fluid from passing through the outer surface 124. However, it should be appreciated that the stent 120 may be covered with standard polyester, ePTFE or other nonporous materials. In other embodiments, the outer surface 124 may be uncovered. In other embodiments, the frame 28 may be self-expanding instead of a balloon-expandable frame.

As shown in FIG. 5, the stent 120 of the inner frame 28 has a diameter 130. As described in greater detail below, the self-expanding or balloon-expandable frame 28 is expandable during implantation from an unexpanded diameter to the expanded diameter 130. In the illustrative embodiment, the expanded diameter 130 is equal to approximately 26 mm when the inner frame 28 is expanded. In the illustrative embodiment, the expanded diameter 130 may be oversized relative to its intended diameter such that an interference fit is created when the device 10 is implanted, as described in greater detail below.

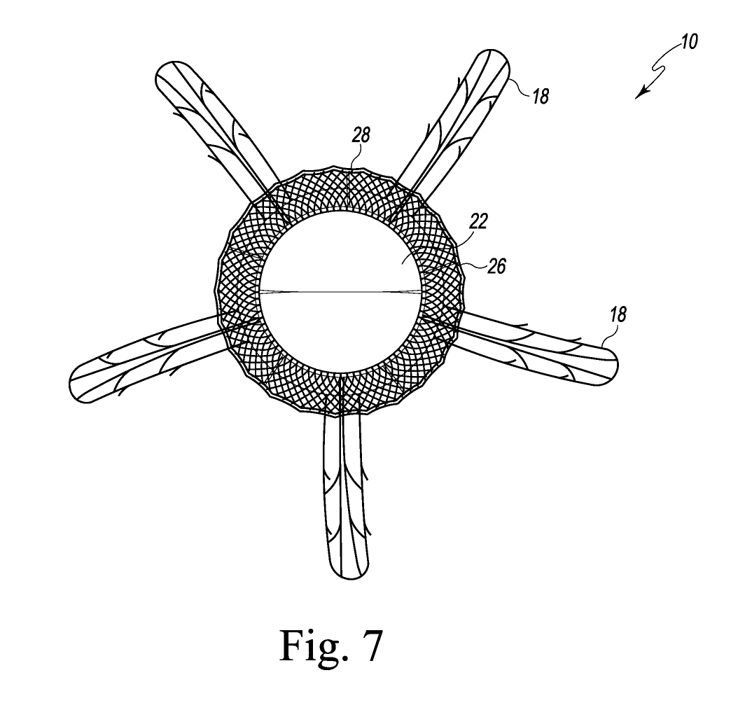

As shown in FIGS. 6-7, the inner frame 28 and the valve 22 are positioned in the passageway 56 defined in the self-expanding outer frame 26. The inner frame 28 has a cranial end 132 that includes the cranial end 14 of the device 10 and a caudal end 134 positioned adjacent to the caudal end 16 of the device 10. As shown in FIG. 6, a distance 136 is defined between the cranial end 132 of the inner frame 28 and the end 44 (see also FIG. 2) of the outer frame 26. In the illustrative embodiment, the distance 136 is equal to 15 mm. In other embodiments, the distance 136 may be greater than or less than 15 mm depending on the patient's anatomy. In other embodiments, the inner frame 28 may be shorted than the outer frame 26.

Additionally, when the inner frame 28 is unexpanded, the outer surface 124 of the inner frame 28 is spaced apart from the outer frame 26. A gap 140 is defined between the outside of the inner frame 28 and the inside of the outer frame 26. In the illustrative embodiment, the gap 140 has a magnitude of about 2 mm to about 3 mm.

As shown in FIG. 8, the device 10 may be implanted in a patient's heart 150. The device 10 may be inserted into the annulus 152 defined between the left atrium 154 and the left ventricle 156 to replace the patient's existing mitral valve. When implanted as shown in FIG. 8, the tips 106 of the barbs 20 of the device 10 engage the walls 158 defining the annulus 152, thereby stabilizing the device 10 in the annulus 152.

In the illustrative embodiment, the diameter 50 of the outer frame 26 is oversized relative to the diameter of the annulus 152. As such, when the device 10 is implanted, the outer frame 26 is reduced to the match the diameter of the annulus 152. The outer surface 124 (FIG. 5) of the inner frame 28 engages the inner surface of the outer frame 26 when the inner frame 28 is expanded. In that way, the gap 140 (FIG. 6) between the frames 26, 28 is closed or reduced and the device 10 is stabilized within the annulus 152 (FIG. 8). It should be appreciated that in other embodiments the inner frame 28 may not engage the inner surface of the outer frame 26.

As described above, the stabilizing arms 18 are biased away from the longitudinal axis 58 of the outer component 36. When the device 10 is implanted as shown in FIG. 8, each stabilizing arm 18 engages the atrial walls 160 of the atrium 154. To provide additional fixation, the barbs 98 extending from each arm 18 are embedded in the walls 160. When positioned as shown in FIG. 8, the valve 22 is located in a supra-annular location within the atrium 154.

In other embodiments, the device may be constructed by adding a separate self-expanding mesh wire to surround the outer component 36 to create stability and seal against the annulus 152. It should also be appreciated that in other embodiments the inner frame is self-expanding and the outer frame may be covered.

In at least one embodiment, a device 10 is implanted into a host tissue structure as shown in FIG. 8. For example, the device 10 can be positionally adjusted, while the inner frame maintains an unexpanded configuration, until a desired placement is achieved according to medical imaging. Upon satisfactory placement, the inner frame 28 can be expanded to bear outward upon the outer frame 26 to further engage the device 10 with host tissue. In a further embodiment, a subsequent second inner frame 28 housing a new valve 22 can be inserted through an originally installed valve, displacing the original leaflets 24 toward the inner side of the original installed valve. Once desired placement of the new valve 22 is achieved, the second inner frame 28 can be expanded outward to engage the originally installed valve from within. Thus, the originally installed inner frame and valve can be functionally replaced by a new inner frame and valve without removal of the original frame and valve. This is particularly advantageous when the original valve 22 and leaflets 24 experience wear from long use.

In at least one embodiment, a device 10 is implanted into a host tissue structure also as shown in FIG. 8. The device 10 is placed into position in an unexpanded configuration of a self-expanding outer frame 26. In a fully expanded configuration, the diameter 50 (FIG. 3) of the outer frame 26 would be oversized relative to the diameter of the annulus 152 (FIG. 8). In such example, the device is implanted in an unexpanded configuration, particularly with regard to minimizing the size of the outer frame 26 around the inner frame 28 during positioning procedures. Upon satisfactory placement, the outer frame 26 is permitted to self-expand against tissue to reach an expanded configuration having a dimension such as diameter 50 that is less than that of the fully expanded configuration. In such state, the outer frame 26 engages and adapts to the dimensions and contours of the host tissue. Furthermore at least in this embodiment, the inner frame 28 has fixed dimensions, such that one size or a small selection of sizes for the inner frame 28 can be applicable for use across a range of patient anatomy dimensions by the advantage of the self-expanding outer frame. Thus, at least in this example, the gap 140 (FIG. 6) increases when the expanded configuration of the device 10 is achieved by permitting self-expansion of the outer frame 26 away from the inner frame 28.

In at least one embodiment, fibers are attached to the outer frame 26 to aid in preventing paravalvular leaks and migration of the device 10 within arterial walls. The fibers in one embodiment include collagen fibers that coat the outer frame 26. The fibers in at least one example extend outwardly from the outer surface 84 of the outer frame 26 to engage host tissue upon implantation, and inwardly into the passageway 56 to engage the inner frame 28. The outer frame 26 may furthermore be covered with hydrogel or other sealing materials. In some embodiments, a plurality of barbs or hooks are attached to the outer frame 26 to further engage tissue and inhibit or prevent migration of the device 10. In an embodiment in which fibers extend inward from the outer frame, the fibers engage the inner frame and create a seal between the inner frame 28 and outer frame 26, particularly when the gap 140 (FIG. 6) is closed or reduced upon expansion of the inner frame 28.

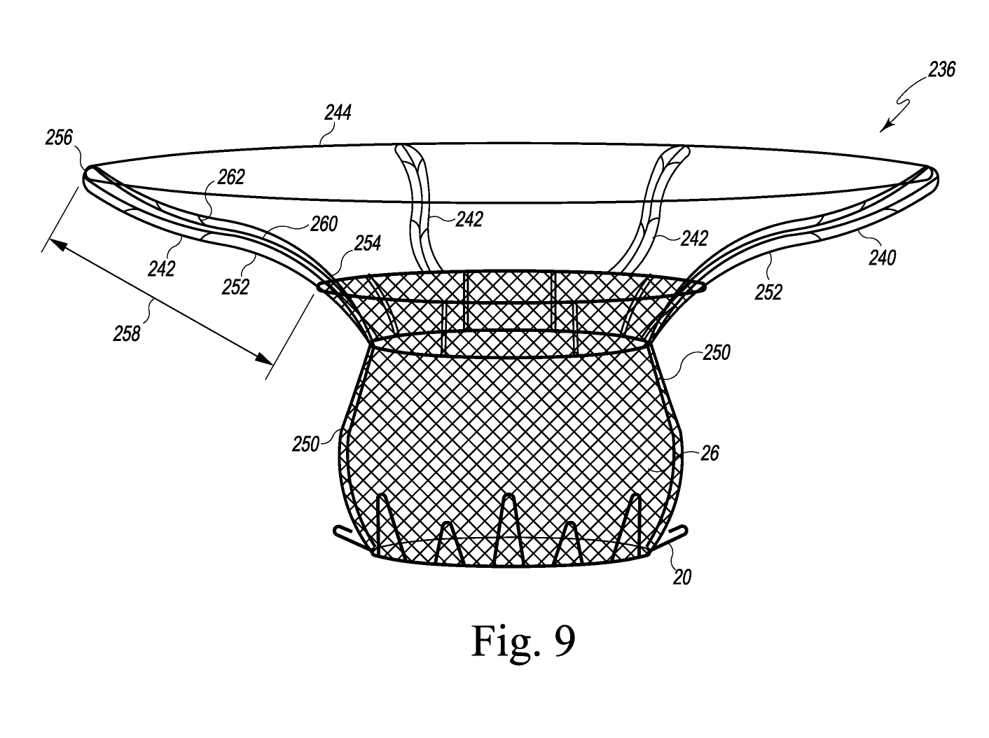

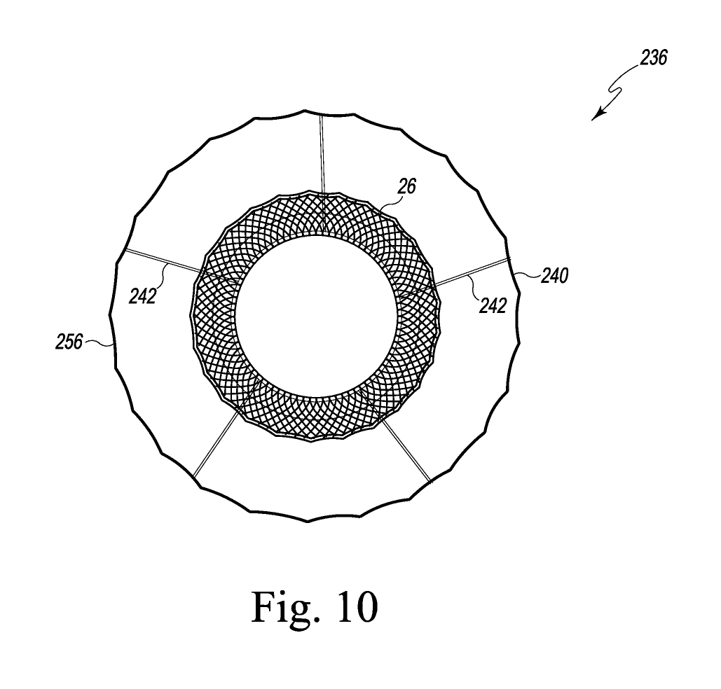

Referring now to FIGS. 9-10, another embodiment of an outer component (hereinafter outer component 236) of the device 10 is shown. Some features of the embodiment illustrated in FIGS. 9-10 are substantially similar to those described above in reference to the embodiment of FIGS. 1-8. Such features are designated in FIGS. 9-10 with the same reference numbers as those used in FIGS. 9-10. Similar to the outer component 36 of FIGS. 1-8, the outer component 236 includes an outer frame 26 and a plurality of barbs 20 extending from the caudal end 16.

As shown in FIGS. 9-10, the outer component 236 also includes a stabilizing frame 240. The stabilizing frame 240 includes a number of arms 242 secured to the outer frame 26 and a ring 244 that joins the arms 242. In the illustrative embodiment, the stabilizing frame 240 is funnel-shaped and is configured to engage the atrial wall of the heart. The frame 240 is formed from a metallic material, such as, nitinol, stainless steel, or other implant grade metallic material. It should be appreciated that in other embodiments the frame 240 may be formed from a polymeric material.

Each arm 242 of the frame 240 includes a base 250 secured to the outer surface of the outer frame 26 and a beam 252 extending outwardly from the base 250. The base 250 of each arm 18 is secured to the outer frame 26 via welding, but it should be appreciated that in other embodiments the bases 250 may be secured to the outer frame 26 by a suture technique or other fastening means.

As shown in FIGS. 9-10, the beam 252 of each stabilizing arm 242 is S-shaped and extends from an end 254 positioned adjacent to an annular flange of the outer frame 26 to a tip 256. A length 258 is defined between the end 254 and the tip 256. As shown in FIG. 9, the length 258 of each beam 252 is equal to approximately 25 mm. In the illustrative embodiment, the tip 256 of each beam 252 is relatively blunt to lessen the risk of injury to the atrial wall. The tips 256 of the beams 252 are joined together by the ring 244.

As shown in FIGS. 9-10, the ring 244 is substantially circular. In other embodiments, the ring 244 may be oval or other geometric shape. Each beam 252 includes a number of longitudinal wires 260 and a number of traverse wires 262 that strengthen each beam 252. In the illustrative embodiment, the stabilizing frame 240 is uncovered, thereby avoiding potential obstruction of a patient's pulmonary veins.

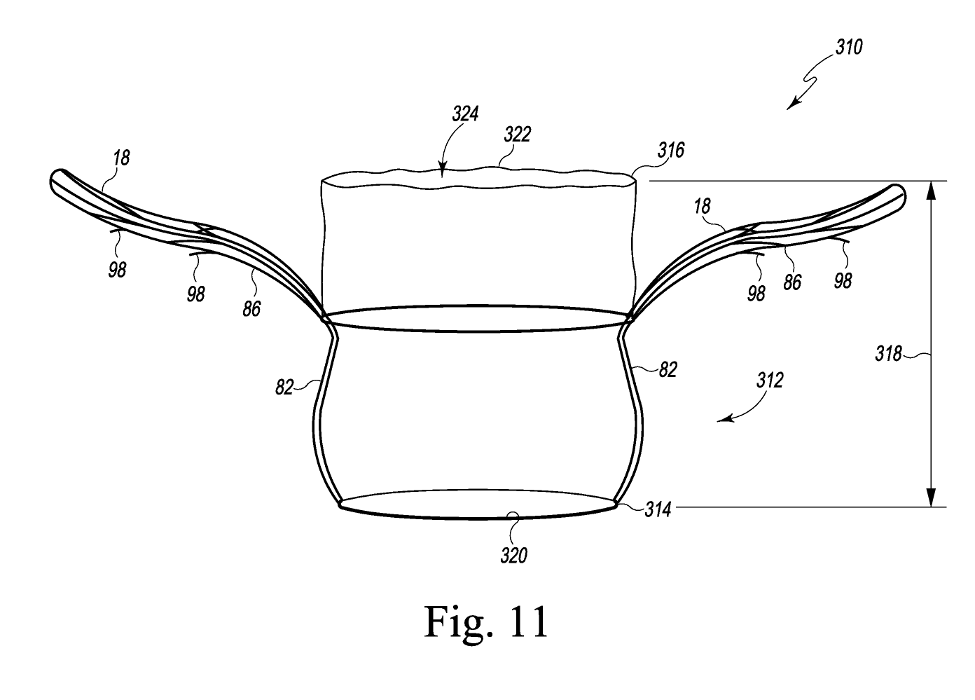

Referring now to FIG. 11, another device (hereinafter device 310) for transcatheter mitral valve repair and replacement is shown. The device 310 includes a self-expanding frame 312 and a plurality of stabilizing arms 18 extending outwardly from the frame 312. In the illustrative embodiment, the frame 312 is formed from a metallic material, such as, nitinol, stainless steel, or other implant grade metallic material. It should be appreciated that in other embodiments the frame 312 may be formed from a polymeric material.

The frame 312 extends from a caudal end 314 to a cranial end 316, and a height 318 is defined therebetween. In the illustrative embodiment, the height 318 is equal to 35 mm. An opening 320 is defined at the caudal end 314 and another opening 322 is defined at the cranial end 316. A passageway 324 extends between the openings 320, 322. In the illustrative embodiment, the passageway 324 is sized to receive an inner component (not shown) including a valve such as, for example, the valve 22 described above in regard to FIGS. 1-8.

As shown in FIG. 11, the stabilizing arms 18 are similar to those described above in regard to FIGS. 1-8. Each stabilizing arm 18 includes a base 82 and a beam 86 extending outwardly from the base 82. The base 82 is secured to the frame 312 via welding but may be secured to the frame 312 by other fastening means. As described above, each beam 86 is configured to engage the atrial wall of a patient's heart and includes a plurality of barbs 98.

To implant the devices 10, 310, a transapical or transvenous (common femoral vein (CFV), or the internal jugular vein (IJV)) trans-septal route may be used. In the transvenous route, the CFV or the IJV are punctured. After placement of a sheath, a guide wire is manipulated into the right atrium (RA). Using a transeptal sheath and needle, the interatrial septum is punctured and a wire passed across into the left ventricle (LV). Using a balloon over the guidewire, the passage in the interatrial septum is dilated to accommodate for the passage of large bore sheaths. The delivery system containing the device 10 is passed through the venous system, across the interatrial septum and positioned across the mitral valve. The deployment sequence is described below.

The transapical route may be utilized for direct access to the LV apex through a small left thoracotomy. The LV apex is punctured and a 7 Fr sheath is introduced into the LV cavity. A soft wire is passed into LA through the mitral valve in a retrograde fashion. A 7 Fr straight catheter is passed into the LA over the wire. The soft wire is now removed and using an introducer, the multi-tip stabilizing wire is introduced into the LA. This wire is stiff and consists of a long common shaft. The distal 15 cm, the wire branches into 4-5 curved tips with varying stiffness, the softest portion being the curved distal portion of the wires. The varying degrees of wire stiffness is demonstrated in the below image with the most distal tip being curved and floppy, transitioning into a straight segment with increasing degrees of stiffness from distal to proximal. This wire will help center itself and the device 10 or the device 310 during the process of deployment. Now the delivery system containing the device 10 or the device 310 may be introduced over this wire across the mitral valve.

The delivery system may include an inner rod, inner cannula and a hydrophilic wire reinforced sheath. The most distal part of the inner rod is connected to a plastic cap measuring 1-1.5 cm in diameter with a hollow interior to accommodate the most cranial aspect of the device 10. In cases where a balloon-expandable stent is used as the inner frame, a balloon will be mounted on the inner rod. The device is mounted on the inner rod and a trigger wire is passed through the inner cannula to traverse the bottom of the outer component 36 of the device 10, passing back into the inner rod and out through the handle of the delivery system. This trigger wire adds additional operator control to the delivery of the device 10. The device is now partially loaded into the sheath utilizing a cone shaped “feeder mechanism” so that the valve 22 of the device 10 is still outside the sheath. In this state of partial loading, the device 10 may be placed in plastic packaging surrounded by preservative fluid, omitting the step of back table loading the device by each individual operator. In this fashion the device and delivery system will be able to be stored together and used off the shelf. The “feeder mechanism” is composed of plastic in a cone shape but with the tip of the cone cut off so that the tip of the cone fits inside the distal opening of the sheath.

After the first centimeter of the apical portion of the cone, there are gaps between the longitudinal parts of the cone. Also the apical portion is made in such a way that the longitudinal component may be peeled off the cone feeder system after the device is fully loaded into the sheath. This can be accomplished by creating weak lines or multiple holes in the plastic of the apical portion to allow easy disassembly of the cone feeder system. The longitudinal portions and the gaps allow the operator to squeeze the cone and help the operator easily complete the loading process of the device.

During the delivery of the device 10, the device 10 is unsheathed to the cylindrical portion of the outer frame within the native mitral valve. At this time the device 10 is still captured by the top cap and the trigger wire. Once the operator is satisfied with the positioning of the device 10 (based on echocardiogram and ventriculogram), the trigger wire is removed and the entire device is unsheathed. Now rapid ventricular pacing (RVP) is performed. During the RVP period, the top cap is pushed cranially to liberate the A components and to stabilize the device within the LA. Next the inner frame containing the valve is deployed within the native mitral annulus to its predetermined diameter. The delivery system and the wires are now removed after a satisfactory echocardiogram and the ventricular apex is repaired.

In other embodiments, a transvenous delivery system may be used. The transvenous system may be very similar to the transapical system described above. In the transvenous system, the device 10 is loaded with its caudal end 16 towards the tip of the delivery system and the top cap does not capture any portion of the device. A trigger wire passes through the tip of the arms 18 and keeps the components folded upwards so the repositioning of the valve is possible after unsheathing. The delivery of the device 10 starts by the transeptal passage of the device and positioning of the device within the native mitral valve. The device 10 is unsheathed. At this time the apparatus may still be moved and repositioned. Once the optimal positioning has been confirmed by echocardiogram and ventriculogram, the trigger wire is removed and the arms 18 of the device 10 may be deployed to stabilize the device within the LA. Next the inner component 38 may be deployed by inflating the delivery system balloon to its predetermined diameter in an example where the inner frame for example is balloon expandable. In an example where the inner frame is self-expanding, the inner frame opens and the valve becomes functional upon withdrawal of the delivery system. The delivery system may be now removed and the transeptal defect is closed with a closure device.

Particular embodiments and features have been described with reference to the drawings. It is to be understood that these descriptions are not limited to any single embodiment or any particular set of features, and that similar embodiments and features may arise or modifications and additions may be made without departing from the scope of these descriptions and the spirit of the appended claims.

1-20. (canceled)

21. A device for placement in a biological tissue fluid conduit comprising:
an anchor frame circumferentially surrounding a longitudinal axis and defining an interior passageway, the anchor frame having an interior side facing the longitudinal axis and passageway, and an exterior side opposite the interior side, the anchor frame configured for engaging with inner facing walls of a biological tissue to anchor the device thereabout;
a valve housing circumferentially surrounding the longitudinal axis; and
a valve housed by the valve housing and configured to restrict fluid flow through the passageway in at least one of two longitudinal directions.

22. A device according to claim 21, wherein the anchor frame is self-expanding.

23. A device according to claim 21, wherein the anchor frame comprises:
a tubular body section surrounding the longitudinal axis and having a longitudinal first end extending in a longitudinal first direction and a longitudinal second end extending in a longitudinal second direction opposite the first direction; and
a proximal section having a longitudinal first end joined to the second end of the body section and a longitudinal second end extending radially away from the longitudinal axis and widening in the second direction.

24. A device according to claim 21, wherein the anchor frame comprises:
a tubular body section surrounding the longitudinal axis and having a longitudinal first end extending in a longitudinal first direction and a longitudinal second end extending in a longitudinal second direction opposite the first direction; and
subvalvular anchors extending from the first end of the body section at least radially away from the longitudinal axis.

25. A device according to claim 24, wherein the anchors extend radially away from the longitudinal axis and in the second direction.

26. A device according to claim 21, further including multiple stabilizer arms extending outward from the anchor frame, wherein each stabilizer arm comprises a spring urged away from the longitudinal axis.

27. A device according to claim 26, wherein each stabilizer arm comprises:
a base secured to the anchor frame and a beam extending from the base and urged away from the longitudinal axis.

28. A device according to claim 27, wherein each at least one beam is configured as a cantilever spring urged away from the longitudinal axis.

29. A device according to claim 26, wherein each stabilizer arm comprises barbs.

30. A device according to claim 29, wherein:
each stabilizer arm comprises a base secured to the anchor frame, a tip opposite the base, and a beam from which barbs extend, the beam extending from the base to the tip; and
the barbs of at least one stabilizer arm are directed at least partially toward the tip thereof.

31. A device according to claim 26, wherein each stabilizer arm comprises an S-shaped portion.

32. A device according to claim 21, wherein the anchor frame defines a proximal end and the valve housing defines a distal end, wherein the proximal end of the anchor frame is engaged with the distal end of the valve housing.

33. A device according to claim 21, wherein the valve housing has a portion received within the anchor frame and a portion extending beyond the anchor frame.

34. A device according to claim 21, wherein the anchor frame and the valve housing are at least partially covered by a fluid impermeable material.

35. A device according to claim 21, wherein the valve leaflets define an opening at about a distal end of the valve housing.

36. A device according to claim 21, wherein an inner diameter of the anchor frame is greater than an inner diameter of the valve housing.

37. A device according to claim 21, wherein a wall of the valve housing has a smaller thickness than a wall of the anchor frame.

38. A device according to claim 21, wherein the anchor frame is formed of a polymeric material.

39. A device according to claim 21, wherein the valve housing is formed of a biological material.

40. A device according to claim 1, wherein the valve housing is formed of a polymeric material.

41. A device according to claim 21, wherein the valve housing is formed of a non-metallic material.

42. A device according to claim 21, wherein the valve housing is free of stent wires.

43. A device according to claim 21, wherein the valve housing is coaxial to and at least partially external to the anchor frame.

44. A device according to claim 21, wherein the valve has commissural attachments and the valve is engaged to the valve housing with the commissural attachments.

45. A device for placement in a biological tissue fluid conduit comprising:
an anchor frame circumferentially surrounding a longitudinal axis and defining an interior passageway, the anchor frame having an interior side facing the longitudinal axis and passageway, and an exterior side opposite the interior side, the anchor frame configured for engaging with inner facing walls of a biological tissue to anchor the device thereabout;
a valve housing circumferentially surrounding the longitudinal axis wherein the valve housing is free from stent wires and formed from non-metallic material; and
a valve housed by the valve housing and configured to restrict fluid flow through the passageway in at least one of two longitudinal directions.
